(19) United States
(12) Patent Application Publication
(10) Pub. No.: US 2024/0188985 A1
(43) Pub. Date: Jun. 13, 2024

(54) LESION RETRIEVAL DEVICE AND ATHERECTOMY DEVICE

(71) Applicant: ASAHI INTECC CO., LTD., Seto-shi (JP)

(72) Inventors: Muneya FURUKAWA, Seto-shi (JP); Kenichi MATSUO, Seto-shi (JP); Akane ISHIKAWA, Seto-shi (JP)

(73) Assignee: ASAHI INTECC CO., LTD., Seto-shi (JP)

(21) Appl. No.: 18/584,340

(22) Filed: Feb. 22, 2024

Related U.S. Application Data
(63) Continuation of application No. PCT/JP22/32173, filed on Aug. 26, 2022.

(30) Foreign Application Priority Data
Aug. 31, 2021 (JP) 2021-140929

(51) Int. Cl. A61B 17/3207

(52) U.S. Cl. CPC A61B 17/320758

(57) ABSTRACT

A lesion retrieval device includes a shaft having an elongated outer shape, a spirally-arranged protruding portion spirally protruding from an outer peripheral surface of the shaft, and an outer cylindrical tube having a hollow cylindrical shape and accommodating, inside thereof, part of a proximal end side of the shaft provided with the spirally-arranged protruding portion. The spirally-arranged protruding portion includes a wire rod that is spirally wound around the outer peripheral surface of the shaft, and a surface of the wire rod in a transverse cross section has irregularities.

### CROSS-REFERENCE TO RELATED APPLICATIONS

This is a Continuation of PCT/JP2022/032173 filed Aug. 26, 2022, which claims priority from JP 2021-140929 filed Aug. 31, 2021. The disclosure of the prior applications is hereby incorporated by reference herein in its entirety.

### TECHNICAL FIELD

The disclosed embodiments relate to a lesion retrieval device and an atherectomy device.

### BACKGROUND

A device that removes a lesion by resecting a stenotic lesion or an obliterative lesion (hereinafter, also collectively referred to simply as a “lesion”) occurring inside a blood vessel has been known. When a lesion tissue is resected by such a device, a tissue fragment constituting the lesion disperses. The dispersed tissue fragment clogs a downstream blood vessel and may contribute to occurrence of a new lesion. Therefore, a device resecting a lesion tissue preferably has a function to suction a tissue fragment into the device and convey same to the outside of the device.

Patent Literature 1 discloses an atheroma excision device that resects a lesion tissue with a tip on the distal end of a shaft by rotating the shaft and suctions a tissue fragment into a tube including, inside thereof, the shaft having a screw thread on an outer peripheral surface of the shaft to convey the tissue fragment. Patent Literatures 2 to 5 each disclose a shaft having a wire rod spirally wound around an outer peripheral surface of the shaft.

### CITATION LIST

### Patent Literature

Patent Literature 1: JP 2015-164529 A
Patent Literature 2: JP 2012-183125 A
Patent Literature 3: US 2016/0101262 A
Patent Literature 4: JP 2015-93122 A
Patent Literature 5: JP 4051292 B

### SUMMARY

### Technical Problem

However, the atheroma excision device described in Patent Literature 1 has had a problem of decrease in efficiency of suctioning a tissue fragment and in efficiency of conveying a tissue fragment, the problem caused by clogging of the tube due to the tissue fragment, depending on the amount of the suctioned tissue fragment or the size of the tissue fragment. In addition, no consideration is given, in each of the shafts disclosed in Patent Literatures 2 to 5, to rotating the shaft, suctioning a tissue fragment into a tube including, inside thereof, the shaft, and conveying the tissue fragment. Such a problem is common to all devices for removing foreign objects inside living body lumens such as the blood vascular system, the lymph gland system, bile tract system, urinary system, respiratory tract system, digestive organ system, secreting gland system, and reproductive organs.

The disclosed embodiments have been made to solve at least part of the above-described problem, and an object of the disclosed embodiments is to provide a lesion retrieval device capable of suppressing decrease in efficiency of suctioning a tissue fragment and in efficiency of conveying a tissue fragment.

### Solution to Problem

The disclosed embodiments have been made to solve at least part of the above-described problem, and can be implemented as the following aspects.

According to one aspect of the disclosed embodiments, a lesion retrieval device is provided. This lesion retrieval device includes a shaft having an elongated outer shape, a spirally-arranged protruding portion spirally protruding from an outer peripheral surface of the shaft, and an outer cylindrical tube having a hollow cylindrical shape and accommodating, inside thereof, part of the proximal end side of the shaft provided with the spirally-arranged protruding portion, wherein the spirally-arranged protruding portion comprises a wire rod that is spirally wound around the outer peripheral surface of the shaft, and a surface of the wire rod in a transverse cross section has irregularities.

### BRIEF DESCRIPTION OF DRAWINGS

FIG. 1 is an explanatory view illustrating a configuration of a lesion retrieval device of a first embodiment.

FIG. 2 is a view explaining a wire rod spirally wound around an outer peripheral surface of a shaft.

FIG. 3 is a view explaining a wire rod spirally wound around an outer peripheral surface of a shaft.

FIG. 4 is an explanatory view showing an example of lesion tissue removal using the lesion retrieval device.

FIG. 5 is an explanatory view explaining conveyance with a spirally-arranged protruding portion.

FIG. 6 is an explanatory view illustrating a configuration of a lesion retrieval device of a second embodiment.

FIG. 7 is an explanatory view explaining conveyance with a spirally-arranged protruding portion.

FIG. 8 is an explanatory view illustrating a configuration of a lesion retrieval device of a third embodiment.

FIG. 9 is a cross-sectional view illustrating a transverse cross section along a line B-B in FIG. 7.

FIG. 10 is an explanatory view illustrating a configuration of a lesion retrieval device of a fourth embodiment.

FIG. 11 is an explanatory view illustrating a configuration of a lesion retrieval device of a fifth embodiment.

FIG. 12 is an explanatory view illustrating a configuration of a lesion retrieval device of a sixth embodiment.

FIG. 13 is an explanatory view illustrating a configuration of a lesion retrieval device of a seventh embodiment.

### DETAILED DESCRIPTION OF EMBODIMENTS

### First Embodiment

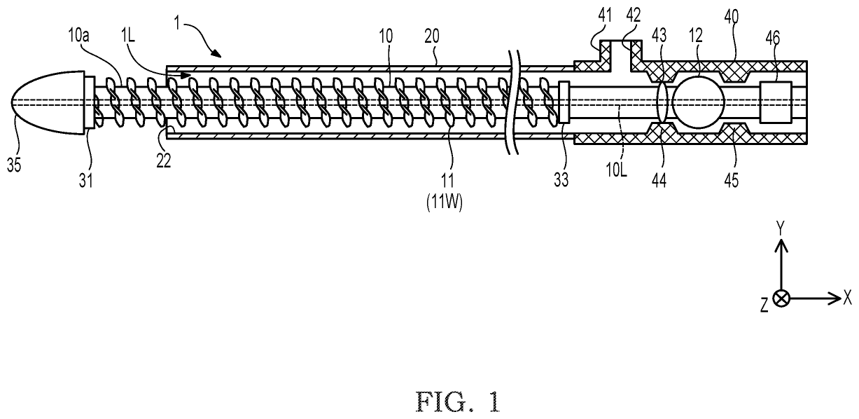

FIG. 1 is an explanatory view illustrating a configuration of a lesion retrieval device 1 of a first embodiment. The lesion retrieval device 1 is a device used when a lesion tissue constituting a stenotic lesion or an obliterative lesion (hereinafter, also collectively referred to simply as a “lesion”) occurring inside a blood vessel is removed, for example. The lesion retrieval device 1 can be configured as a device for removing a lesion tissue (or a foreign object) generated inside a living body lumen such as the coronary artery and other blood vascular systems including the heart, the lymph gland system, bile tract system, urinary system, respiratory tract system, digestive organ system, secreting gland system, and reproductive organs. The lesion retrieval device 1 includes a shaft 10, an outer cylindrical tube 20, a distal end side joint part 31, a proximal end side joint part 33, a tip 35, and a motor housing 40.

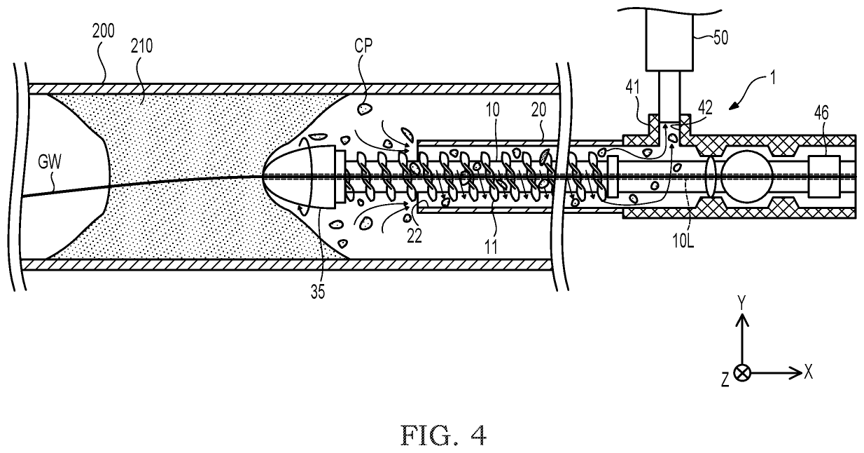

For convenience of explanation, FIG. 1 includes a portion illustrated such that the relative ratios of sizes of respective constituent members are different from actual ratios. In addition, FIG. 1 includes a portion where part of each constituent member is exaggeratedly illustrated. FIG. 1 also shows X-, Y-, and Z-axes orthogonal to one another. The X-axis corresponds to the longitudinal direction of the lesion retrieval device 1, the Y-axis corresponds to the height direction of the lesion retrieval device 1, and the Z-axis corresponds to the width direction of the lesion retrieval device 1. The left side (−X-axis direction) of FIG. 1 is referred to as a “distal end side” of the lesion retrieval device 1 and each constituent member thereof, and the right side (+X-axis direction) of FIG. 1 is referred to as a “proximal end side” of the lesion retrieval device 1 and each constituent member thereof. In addition, one end positioned on the distal end side among the both ends in the longitudinal direction (X-axis direction) of the lesion retrieval device 1 and each constituent member thereof is referred to as a “distal end,” and the other end positioned on the proximal end side is referred to as a “proximal end.” Furthermore, the distal end and a vicinity thereof are referred to as a “distal end portion,” and the proximal end and a vicinity thereof are referred to as a “proximal end portion.” The distal end side is inserted into the inside of a living body, and the proximal end side is operated by an operator such as a medical doctor. These points are also true for FIGS. 4-8 and 10-13.

In FIG. 1, a cross-sectional surface of the outer cylindrical tube 20 cut along the XY plane is illustrated with diagonal hatching, and a cross-sectional surface cut along the XY plane of the motor housing 40 is illustrated with grid hatching. In addition, with respect to components of the lesion retrieval device 1 other than the outer cylindrical tube 20 and the motor housing 40, external appearances are illustrated in FIG. 1 for convenience of explanation. That is, FIG. 1 illustrates the lesion retrieval device 1 in a state where components inside the outer cylindrical tube 20 and the motor housing 40 are viewable from the outside.

The shaft 10 is a member having a long (elongated) outer shape extending along the longitudinal direction of the lesion retrieval device 1. The shaft 10 is provided inside (in an inner cavity 1L) of the outer cylindrical tube 20 and the motor housing 40. The shaft 10 preferably has antithrombogenicity, flexibility, and biocompatibility and can be formed from a resin material or a metal material. As the resin material, for example, a polyamide resin, a polyolefin resin, a polyester resin, a polyurethane resin, a silicone resin, a fluororesin, and the like can be adopted. As the metal material, for example, stainless-steel such as SUS304, a nickel-titanium alloy, a cobalt-chromium alloy, and the like can be adopted. In the present embodiment, the shaft 10 is a multi-thread coil in which a plurality of wires is wound in a multi-thread manner and is composed of stainless-steel such as SUS304.

The shaft 10 has a device lumen 10L inside thereof. As illustrated by the dashed lines in FIG. 1, the device lumen 10L extends along the longitudinal direction (X-axis direction) of the lesion retrieval device 1. A delivery guide wire as a medical device is insertable into the device lumen 10L. Hereinafter, the delivery guide wire is also simply referred to as a “guide wire.” The outer shape and the length of the shaft 10 and the inner diameter of the device lumen 10L can be arbitrarily designed.

A spirally-arranged protruding portion 11 and a large diameter portion 12 are provided on the shaft 10. The spirally-arranged protruding portion 11 spirally protrudes from the outer peripheral surface 10a of the shaft 10. In the spirally-arranged protruding portion 11, a wire rod is spirally wound around the outer peripheral surface 10a of the shaft 10, a surface of the wire rod in a transverse cross section having irregularities. As used herein, “irregularities” in transverse cross section refer to variations in the outer contour. For example, the irregularities in the surface of the wire rod in a transverse cross section may include convex edges, as described below.

Hereinafter, the wire rod spirally wound around the outer peripheral surface 10a of the shaft 10 is referred to as a winding wire rod 11W. In the present embodiment, the spirally-arranged protruding portion 11 is formed by winding, in the Z twist direction, the winding wire rod 11W around the outer peripheral surface 10a of the shaft 10. The large diameter portion 12 is a portion on the proximal end side of the shaft 10, in which the diameter thereof is larger than that of the other portions.

The outer cylindrical tube 20 is a hollow cylindrical member accommodating, inside thereof, part of the proximal end side of the shaft 10 provided with the spirally-arranged protruding portion 11. In other words, the distal end side of the shaft 10 provided with the spirally-arranged protruding portion 11 is exposed from the outer cylindrical tube 20. The spirally-arranged protruding portion 11 provided in the portion of the shaft 10 exposed from the outer cylindrical tube 20 facilitates suction of fluid into the lesion retrieval device 1 from the periphery of the lesion retrieval device 1 on the distal end side, when the shaft 10 and the spirally-arranged protruding portion 11 rotate. An opening 22 is formed at the distal end of the outer cylindrical tube 20. The opening 22 is formed over the periphery of the shaft 10 when the lesion retrieval device 1 is viewed from the distal end side. In the present embodiment, the outer cylindrical tube 20 is formed by causing a hollow cylindrical member formed from tetrafluoroethylene-perfluoroalkyl vinyl ether copolymer (PFA) to thermally shrink. The outer cylindrical tube 20 may be formed from tetrafluoroethylene-hexafluoropropylene copolymer (FEP) or may be formed from another resin material having antithrombogenicity, flexibility, and biocompatibility. In addition, the outer cylindrical tube 20 may be a resin tube including, inside thereof, a metal braid.

The distal end side joint part 31 is a disc-shaped member and joins the distal end portion of the shaft 10 and the proximal end portion of the tip 35. In addition, the proximal end portion of the distal end side joint part 31 is also joined to the distal end portion of the spirally-arranged protruding portion 11. The proximal end side joint part 33 is an annular member into which the shaft 10 is insertable and is fixed to the shaft 10 in a state where the shaft 10 is inserted. In the present embodiment, the position on the shaft 10 in which the proximal end side joint part 33 is fixed corresponds to the proximal end portion of the outer cylindrical tube 20 in the X-axis direction. In addition, the distal end portion of the proximal end side joint part 33 is joined to the proximal end portion of the spirally-arranged protruding portion 11. An arbitrary adhesive, for example, metal solder such as silver solder, gold solder, zinc, Sn—Ag alloy, or Au—Sn alloy or an adhesive such as an epoxy-based adhesive can be adopted for joining with the distal end side joint part 31 and the proximal end side joint part 33.

The tip 35 is a member joined to the distal end portion of the distal end side joint part 31 and advances inside a living body lumen ahead of other members. As illustrated in FIG. 1, the tip 35 has an outer shape in which the diameter thereof decreases from the proximal end side toward the distal end side, in order to enable the lesion retrieval device 1 to smoothly travel inside a living body lumen. When the tip 35 comes into contact with a lesion tissue formed inside a blood vessel, the lesion tissue is resected (see FIG. 4).

The motor housing 40 is a member which has an approximately hollow cylindrical shape and accommodates packing 43 and a motor 46, and the shaft 10 is inserted into the motor housing 40. In addition, the outer cylindrical tube 20 is joined to the distal end portion of the motor housing 40. The motor housing 40 includes a projecting portion 41 projecting in the +Y-axis direction. In addition, a discharge port 42 is formed in the projecting portion 41. The discharge port 42 is an opening for discharging, to the outside of the lesion retrieval device 1, a tissue fragment of a lesion tissue suctioned from the opening 22 into the lesion retrieval device 1 (see FIG. 4).

The packing 43 is an annular member into which the shaft 10 is insertable and is formed from a material such as rubber or various elastomers. The packing 43 is fitted into a distal end side reduced diameter portion 44 in which the diameter thereof reduces due to an inner wall projecting in the inner space of the motor housing 40, and seals the distal end side reduced diameter portion 44 so that fluid does not leak from the distal end side reduced diameter portion 44 to the proximal end side.

A proximal end side reduced diameter portion 45 is a reduced diameter portion formed on the proximal end side from the distal end side reduced diameter portion 44. Movement of the shaft 10 in the longitudinal direction (X-axis direction) of the lesion retrieval device 1 is suppressed by arranging the large diameter portion 12 between the distal end side reduced diameter portion 44 and the proximal end side reduced diameter portion 45.

The motor 46 is a rotation transmission mechanism which is connected to the shaft 10 and transmits rotational force to the shaft 10 and thereby rotates the shaft 10 and the spirally-arranged protruding portion 11 around the axis. In the present embodiment, the motor 46 directly transmits rotational force to the shaft 10. The motor 46 may indirectly transmit rotational force to the shaft 10 via another member. Rotational force may be transmitted to the shaft 10 by manually rotating a handle instead of the motor 46.

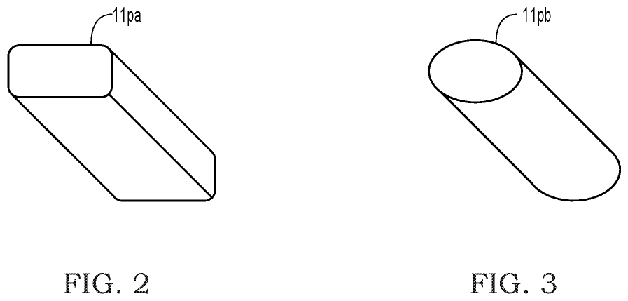

FIGS. 2 and 3 are views explaining the wire rod (winding wire rod 11W) spirally wound around the outer peripheral surface 10a of the shaft 10 as the spirally-arranged protruding portion 11. A base wire rod 11pa illustrated in FIG. 2 is a wire rod having a rectangular transverse cross section. Specifically, since the base wire rod 11pa is a wire rod formed by rolling a round wire having a circular transverse cross section, four corners of the rectangular transverse cross section are rounded. The winding wire rod 11W has a configuration in which the base wire rod 11pa is spirally twisted. In the present embodiment, the base wire rod 11pa is twisted in the S twist direction. The spirally-arranged protruding portion 11 is formed by spirally winding, around the outer peripheral surface 10a of the shaft 10, the base wire rod 11pa in a state of being spirally twisted. As described above, in the present embodiment, as the spirally-arranged protruding portion 11 is wound around the shaft 10 in the Z twist direction, the direction in which the base wire rod 11pa is twisted is different from the winding direction in which the winding wire rod 11W is wound around the shaft 10.

The base wire rod 11pb illustrated in FIG. 3 is a wire rod having an elliptical transverse cross section. The winding wire rod 11W may have a configuration in which the base wire rod 11pb is spirally twisted. Incidentally, the transverse cross section of the base wire rod preferably includes an angulated portion. This is because the angulated portion of the base wire rod on the transverse cross section forms a convex shape on the surface of the winding wire rod 11W when the base wire rod is twisted, and the convex shape is easily sharpened through extension along the twisting direction when the base wire rod is twisted. Such a portion having a sharpened convex shape contributes to shredding of a tissue fragment suctioned into the outer cylindrical tube 20 from the opening 22.

FIG. 4 is an explanatory view showing an example of lesion tissue removal using the lesion retrieval device 1 of the present embodiment. FIG. 4 exemplifies a blood vessel 200 as an example of the living body lumen and exemplifies a lesion tissue 210 as an example of the lesion tissue. A lesion obliterated with the lesion tissue 210 is formed in the blood vessel 200 shown in FIG. 4.

When the lesion tissue is removed using the lesion retrieval device 1, an operator firstly delivers a guide wire GW so that a distal end portion of the guide wire GW is positioned on the proximal end side of the lesion tissue 210. Then, the operator inserts, from the distal end side of the device lumen 10L, the guide wire GW into the device lumen 10L, with the proximal end side of the guide wire GW in the lead. After inserting the guide wire GW, the operator pushes the lesion retrieval device 1 forward along the guide wire GW and delivers the lesion retrieval device 1 until the tip 35 reaches the position of the lesion tissue 210.

After the lesion retrieval device 1 reaches the position of the lesion tissue 210, the operator rotates the shaft 10 and the spirally-arranged protruding portion 11 by driving the motor 46. When the tip 35 rotating together with the shaft 10 is pressed against the lesion tissue 210, the lesion tissue 210 is resected, and a tissue fragment CP is dispersed. In addition, also in the case where the tip 35 is pressed against the lesion tissue 210 in a state where the spirally-arranged protruding portion 11 is exposed from the outer cylindrical tube 20, the lesion tissue 210 is resected, and a tissue fragment CP is dispersed. In a state where the shaft 10 and the spirally-arranged protruding portion 11 rotate, the distance between the lesion tissue 210 and the lesion retrieval device 1 is lengthened or shortened, if needed. The tissue fragment CP is suctioned into the outer cylindrical tube 20 via the opening 22 as the spirally-arranged protruding portion 11 rotates, and then is conveyed, by the spirally-arranged protruding portion 11 functioning as an Archimedean screw, toward the proximal end side of the lesion retrieval device 1. The tissue fragment CP being conveyed is shredded by the convex-shaped portion forming an edge among the irregularities on the surface of the spirally-arranged protruding portion 11. The tissue fragment CP conveyed while being shredded in this manner is discharged to the outside of the lesion retrieval device 1 from the discharge port 42. In FIG. 4, a suction part 50 is connected to the discharge port 42, and the tissue fragment CP reaching the discharge port 42 is suctioned into the suction part 50.

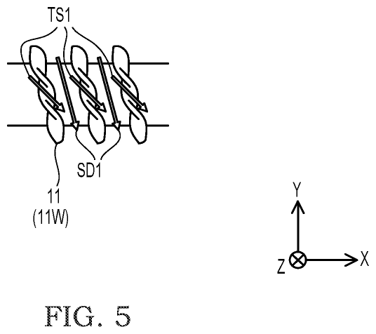

FIG. 5 is an explanatory view explaining conveyance with the spirally-arranged protruding portion 11. As described above, the spirally-arranged protruding portion 11 is formed by winding the winding wire rod 11W, which has a configuration in which the base wire rod 11pa is twisted in the S twist direction, in the Z twist direction with respect to the shaft 10, in the lesion retrieval device 1. Since the spirally-arranged protruding portion 11 is formed as described above, the spiral direction SD1 in which the spirally-arranged protruding portion 11 extends and the extension direction TS1 in which the portion having a convex shape on the surface of the spirally-arranged protruding portion 11 extends both face the proximal end side (+X-axis direction). As a result, when the spirally-arranged protruding portion 11 rotates, a fluid flow along the extension direction TS1 is merged to a fluid flow along the spiral direction SD1, and suction of a tissue fragment toward the proximal end side is thereby facilitated.

As described above, according to the lesion retrieval device 1 of the first embodiment, the portion of the shaft 10 accommodated in the outer cylindrical tube 20 is provided with the spirally-arranged protruding portion 11. Therefore, a tissue fragment suctioned into the outer cylindrical tube 20 can be conveyed by the spirally-arranged protruding portion 11 functioning as an Archimedean screw inside the outer cylindrical tube 20, as the shaft 10 and the spirally-arranged protruding portion 11 rotate. In addition, according to this configuration, the winding wire rod 11W is wound around the outer peripheral surface 10a of the shaft 10 in the spirally-arranged protruding portion 11, a surface of the winding wire rod 11W on a transverse cross section having irregularities. Therefore, the portion having a convex shape among the irregularities on the surface of the spirally-arranged protruding portion 11 forms an edge, making it possible to shred a tissue fragment being conveyed inside the outer cylindrical tube 20. Therefore, clogging due to the tissue fragment suctioned into the outer cylindrical tube 20 can be suppressed, and decrease in efficiency of suctioning a tissue fragment and in efficiency of conveying a tissue fragment can be thus suppressed.

According to the lesion retrieval device 1 of the first embodiment, the winding wire rod 11W has a structure in which the base wire rod 11pa having a rectangular transverse cross section is spirally twisted, the irregularities on the surface on a transverse cross section of the winding wire rod 11W can be easily achieved thereby. In addition, since such a winding wire rod 11W is wound around the outer peripheral surface 10a of the shaft 10 to form the spirally-arranged protruding portion 11, the spirally-arranged protruding portion 11 is easily achieved. In addition, according to this configuration, the direction in which the base wire rod 11pa is twisted is different from the winding direction in which the winding wire rod 11W is wound around the shaft 10. Therefore, the spiral direction SD1 in which the spirally-arranged protruding portion 11 extends and the extension direction TS1 in which the portion having a convex shape on the surface of the spirally-arranged protruding portion 11 extends both face the proximal end side, and a fluid flow along the spiral direction SD1 and a fluid flow along the extension direction TS1 are thus merged. As a result, conveyance of a tissue fragment toward the proximal end side of the lesion retrieval device 1 can be facilitated.

Furthermore, according to the lesion retrieval device 1 of the first embodiment, since the shaft 10 is a multi-thread coil in which a plurality of wires is wound in a multi-thread manner, torquability and flexibility of the shaft 10 can be improved.

Furthermore, according to the lesion retrieval device 1 of the first embodiment, since the shaft 10 has, inside thereof, the device lumen 10L into which a medical device is insertable, the lesion retrieval device 1 can be easily delivered to a lesion by inserting a delivery guide wire into the device lumen 10L.

Furthermore, according to the lesion retrieval device 1 of the first embodiment, since the motor 46 as the rotation transmission mechanism rotating the shaft 10 and the spirally-arranged protruding portion 11 around the axis is provided, the shaft 10 and the spirally-arranged protruding portion 11 can be easily rotated using the rotation transmission mechanism.

### Second Embodiment

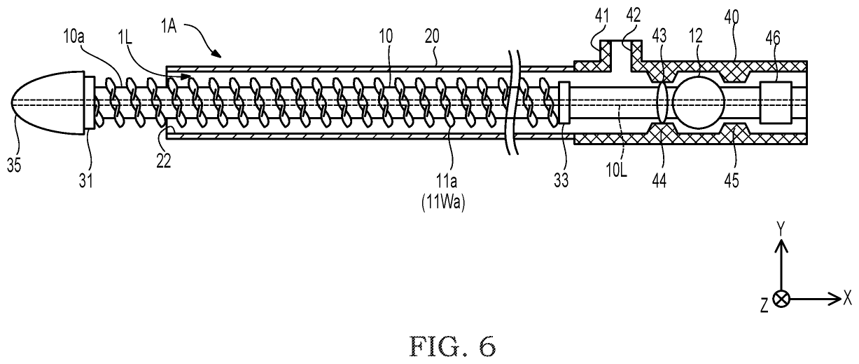

FIG. 6 is an explanatory view illustrating a configuration of a lesion retrieval device 1A of a second embodiment. The lesion retrieval device 1A of the second embodiment has the same configuration as the lesion retrieval device 1 of the first embodiment except that a spirally-arranged protruding portion 11a is provided instead of the spirally-arranged protruding portion 11 of the first embodiment.

The spirally-arranged protruding portion 11a is formed by winding, around the outer peripheral surface 10a of the shaft 10 in the Z twist direction, the base wire rod 11pa (winding wire rod 11Wa) in a state of being spirally twisted, in a manner similar to the spirally-arranged protruding portion 11 of the first embodiment. On the other hand, in the spirally-arranged protruding portion 11a, the base wire rod 11pa (winding wire rod 11Wa) is twisted in the Z twist direction unlike the spirally-arranged protruding portion 11 of the first embodiment. Accordingly, in the second embodiment, the direction in which the base wire rod 11pa is twisted is identical to the winding direction in which the winding wire rod 11Wa is wound around the shaft 10.

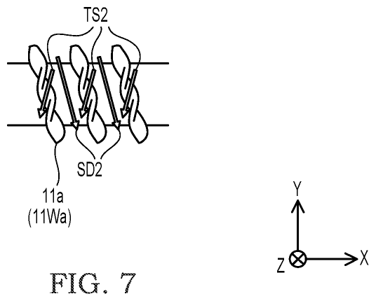

FIG. 7 is an explanatory view explaining conveyance with the spirally-arranged protruding portion 11a. As described above, the spirally-arranged protruding portion 11a is formed by winding the winding wire rod 11Wa, which has a configuration in which the base wire rod 11pa is twisted in the Z twist direction, in the Z twist direction with respect to the shaft 10, in the lesion retrieval device 1A. Since the spirally-arranged protruding portion 11a is formed as described above, the spiral direction SD2 in which the spirally-arranged protruding portion 11a extends faces the proximal end side (+X-axis direction), and the extension direction in which the portion having a convex shape on the surface of the spirally-arranged protruding portion 11a extends faces the distal end side (−X-axis direction). As a result, when the spirally-arranged protruding portion 11a rotates, shear force is easily generated between fluid flowing along the spiral direction SD2 and fluid flowing along the extension direction TS2. Such shear force contributes to shredding of a tissue fragment in addition to the convex-shaped portion forming an edge on the surface of the spirally-arranged protruding portion 11, and shredding of the tissue fragment is thus facilitated.

The same effect as the first embodiment can also be provided by the lesion retrieval device 1A of the second embodiment described above. In addition, according to the lesion retrieval device 1A of the second embodiment, shear force is easily generated between fluid flowing along the spiral direction SD2 and fluid flowing along the extension direction TS2, as described above, and shredding of a tissue fragment can thus be facilitated.

### Third Embodiment

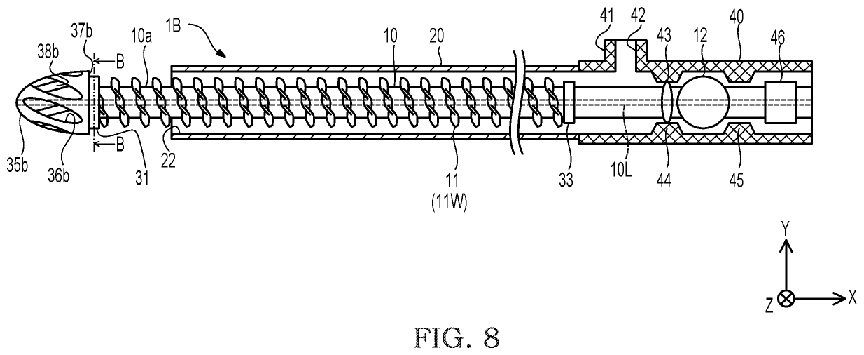

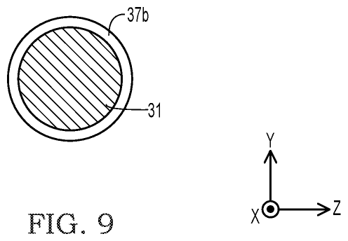

FIG. 8 is an explanatory view illustrating a configuration of a lesion retrieval device 1B of a third embodiment. FIG. 9 is a cross-sectional view illustrating a transverse cross section along a line B-B in FIG. 8. The lesion retrieval device 1B of the third embodiment has the same configuration as the lesion retrieval device 1 of the first embodiment except that a cutter assembly 35b is provided instead of the tip 35 of the first embodiment. The lesion retrieval device 1B provided with the cutter assembly 35b corresponds to an atherectomy device.

The cutter assembly 35b is joined to the distal end portion of the shaft 10 via the distal end side joint part 31. The cutter assembly 35b is a member having an outer shape in which the diameter thereof decreases from the proximal end side toward the distal end side. The cutter assembly 35b has a peripheral surface opening 36b and a proximal end side opening 37b and accommodates, inside thereof, a cutter 38b capable of resecting a body tissue. The peripheral surface opening 36b is an opening provided on a peripheral surface of the cutter assembly 35b. The proximal end side opening 37b is an opening provided around the periphery of the distal end side joint part 31 and faces the proximal end side (see FIG. 9). The cutter assembly 35b rotates with rotation of the shaft 10. In the third embodiment, when a lesion tissue formed in a blood vessel is removed, the cutter assembly 35b is pressed against the lesion tissue in a state where the shaft 10 is rotating. At this time, the lesion tissue (or a tissue fragment dispersed from the lesion tissue) having entered the cutter assembly 35b from the peripheral surface opening 36b is shredded by the cutter 38b and discharged to the outside of the cutter assembly 35b from the proximal end side opening 37b. The tissue fragment of the lesion tissue discharged from the proximal end side opening 37b is suctioned into the outer cylindrical tube 20 via the opening 22 as the spirally-arranged protruding portion 11 rotates.

The same effect as the first embodiment can also be provided by the lesion retrieval device 1B of the third embodiment described above. In addition, according to the lesion retrieval device 1B of the third embodiment, since the cutter 38b capable of excising a body tissue is provided at the distal end portion of the shaft 10, a lesion tissue can be efficiently resected, and a lesion tissue can be resected even in a case of hard lesion such as a calcified lesion. In addition, in a case where the lesion tissue formed in a blood vessel is hard, since the lesion tissue is suctioned into the outer cylindrical tube 20 after shredding the lesion tissue with the cutter 38b, decrease in suctioning efficiency and in conveying efficiency can be suppressed when a hard lesion tissue is removed.

### Fourth Embodiment

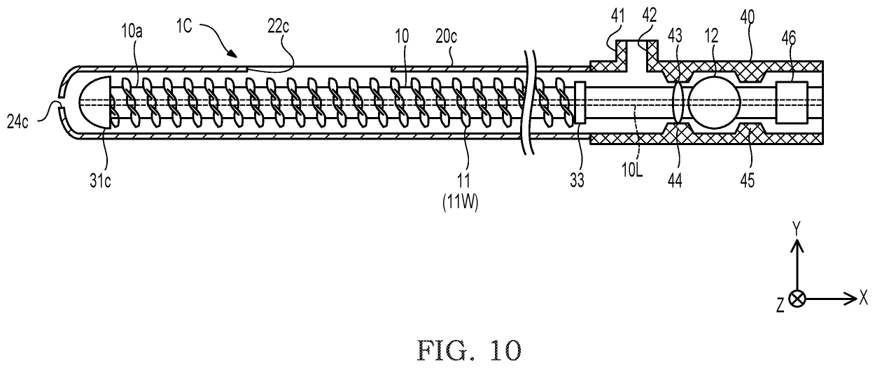

FIG. 10 is an explanatory view illustrating a configuration of a lesion retrieval device 1C of a fourth embodiment. The lesion retrieval device 1C of the fourth embodiment has the same configuration as the lesion retrieval device 1 of the first embodiment except that an outer cylindrical tube 20c is provided instead of the outer cylindrical tube 20 of the first embodiment, and that a distal end side joint part 31c is provided instead of the distal end side joint part 31 of the first embodiment. An opening 22c and an opening 24c are formed on the outer cylindrical tube 20c instead of the opening 22.

The opening 22c is formed along the longitudinal direction (X-axis direction) of the lesion retrieval device 1 on the outer peripheral surface of the outer cylindrical tube 20c. The opening 22c is an opening for suctioning a tissue fragment into the outer cylindrical tube 20c when the spirally-arranged protruding portion 11 rotates. Although the opening 22c is formed in the +Y-axis direction in FIG. 10, the opening 22c may be formed at any positions on the outer peripheral surface of the outer cylindrical tube 20c, and two or more openings 22c may be formed.

The opening 24c is formed at the distal end of the outer cylindrical tube 20c. The opening 24c is formed at a position which is an extension of the device lumen 10L on the distal end side (−X-axis direction side) of the shaft 10. The opening 24c is an opening used as an inlet through which a guide wire is inserted into the device lumen 10L when a lesion tissue is removed.

The distal end side joint part 31c is a hemispherical member and is joined to the distal end portion of the shaft 10. In addition, a proximal end portion of the distal end side joint part 31c is joined to the distal end portion of the spirally-arranged protruding portion 11. In the fourth embodiment, when a lesion tissue formed in a blood vessel is removed, the distal end portion of the outer cylindrical tube 20c is pressed against the lesion tissue 210 to resect the lesion tissue 210, and a tissue fragment dispersed is suctioned into the lesion retrieval device 1 through the opening 22c.

The same effect as the first embodiment described above can also be provided by the lesion retrieval device 1C of the fourth embodiment. In addition, according to the lesion retrieval device 1C of the fourth embodiment, the opening 22c is formed along the longitudinal direction (X-axis direction) of the lesion retrieval device 1 on the outer peripheral surface of the outer cylindrical tube 20c. Therefore, the opening 22c can be formed so as to be larger than that in the case where an opening is formed in the distal end portion of the outer cylindrical tube 20c. The larger the opening 22c is, the easier a tissue fragment is suctioned into the lesion retrieval device 1C, and efficiency of suctioning a tissue fragment can thus be improved.

### Fifth Embodiment

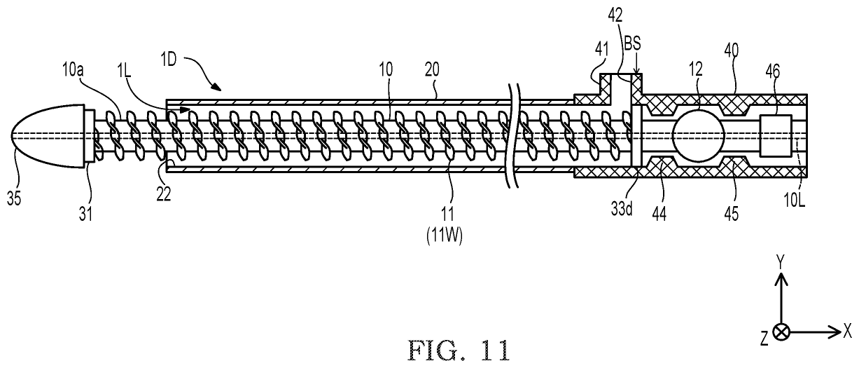

FIG. 11 is an explanatory view illustrating a configuration of a lesion retrieval device 1D of a fifth embodiment. The lesion retrieval device 1D of the fifth embodiment has the same configuration as the lesion retrieval device 1 of the first embodiment except that a proximal end side joint part 33d is provided instead of the proximal end side joint part 33 of the first embodiment and that the packing 43 of the first embodiment is not provided.

The proximal end side joint part 33d is an annular member into which the shaft 10 is insertable and is joined to the proximal end portion of the spirally-arranged protruding portion 11. The proximal end side joint part 33d is fixed in a portion on the shaft 10 corresponding to a proximal end portion BS of the projecting portion 41 in the X-axis direction in a state where the shaft 10 is inserted. Since the proximal end side joint part 33d rotates with rotation of the shaft 10, the proximal end side joint part 33d cannot be fitted into the inner side of the motor housing 40; however, it is preferable that the cross-section formed in the YZ plane be large so as to prevent fluid from flowing toward the proximal end side.

The same effect as the first embodiment described above can also be provided by the lesion retrieval device 1D of the fifth embodiment. In addition, since the lesion retrieval device 1D of the fifth embodiment includes the proximal end side joint part 33d, fluid can be prevented from flowing toward the proximal end side even without packing.

### Sixth Embodiment

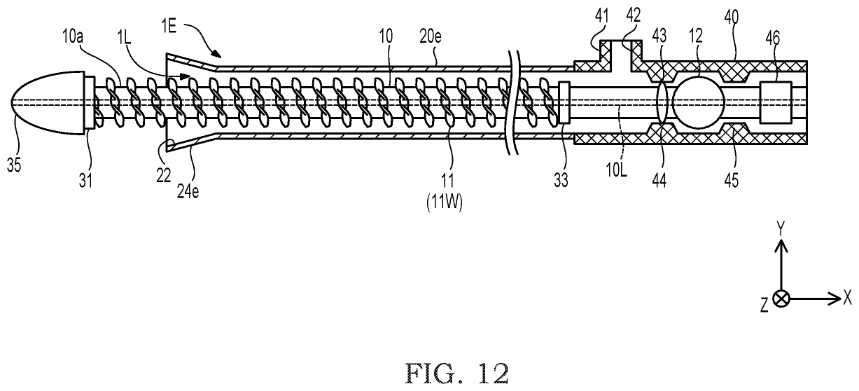

FIG. 12 is an explanatory view illustrating a configuration of a lesion retrieval device 1E of a sixth embodiment. The lesion retrieval device 1E of the sixth embodiment has the same configuration as the lesion retrieval device 1 of the first embodiment except that an outer cylindrical tube 20e is provided instead of the outer cylindrical tube 20 of the first embodiment. A large diameter portion 24e is formed in the outer cylindrical tube 20e. The large diameter portion 24e is formed in the distal end portion of the outer cylindrical tube 20e and is a portion in which the diameter of the outer cylindrical tube 20e is increased toward the distal end side.

The same effect as the first embodiment described above can also be provided by the lesion retrieval device 1E of the sixth embodiment. In addition, according to the lesion retrieval device 1E of the sixth embodiment, since the large diameter portion 24e is formed in the outer cylindrical tube 20e, the opening 22 formed in the distal end of the outer cylindrical tube 20e can be made large. As the size of the opening 22 formed becomes large, the possibility to suction a tissue fragment into the lesion retrieval device 1 increases, and efficiency of suctioning a tissue fragment into the lesion retrieval device 1 can thus be improved.

### Seventh Embodiment

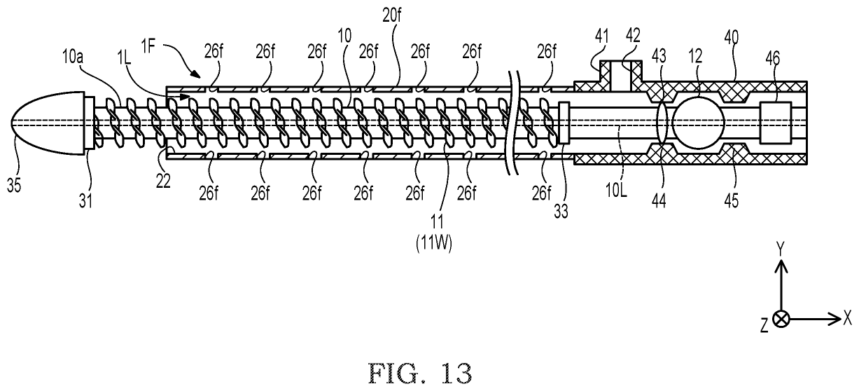

FIG. 13 is an explanatory view exemplifying a configuration of a lesion retrieval device 1F of a seventh embodiment. The lesion retrieval device 1F of the seventh embodiment has the same configuration as the lesion retrieval device 1 of the first embodiment except that an outer cylindrical tube 20f is provided instead of the outer cylindrical tube 20 of the first embodiment. A plurality of openings 26f is formed on the outer cylindrical tube 20f. The plurality of openings 26f is formed on the outer peripheral surface of the outer cylindrical tube 20 and each opening 26f is smaller than the opening 22.

The same effect as the first embodiment described above can also be provided by the lesion retrieval device 1F of the seventh embodiment. In addition, according to the lesion retrieval device 1F of the seventh embodiment, the outer cylindrical tube 20f has the plurality of openings 26f. A tissue fragment suctioned into the outer cylindrical tube 20 from the opening 22 is sequentially shredded by the spirally-arranged protruding portion 11 as the tissue fragment travels toward the proximal end side. At this time, when the opening area of the openings 26f is relatively small, the tissue fragment cannot pass the openings 26f, and blood is introduced into the inside of the outer cylindrical tube 20 from the openings 26f as the spirally-arranged protruding portion 11 rotates. Since the viscosity of a tissue fragment is high, the viscosity of fluid flowing inside the outer cylindrical tube 20 tends to be also increased due to accumulation of the tissue fragment inside the outer cylindrical tube 20; however, the viscosity of such fluid can be decreased by blood introduced. Accordingly, when the opening area of the openings 26f is set to be relatively small, decrease in efficiency of conveying a tissue fragment can be prevented.

On the other hand, in the case where the opening area of the openings 26f is relatively large, the openings 26f can serve as an inlet when a tissue fragment generated by resecting a lesion tissue and dispersed into a blood vessel is suctioned into the outer cylindrical tube 20, similar to the opening 22. That is, a tissue fragment can be suctioned into the outer cylindrical tube 20 also from the openings 26f in addition to the opening 22 when the spirally-arranged protruding portion 11 is rotating. Accordingly, when the opening area of the openings 26f is set to be relatively large, efficiency of collecting a tissue fragment dispersed into a blood vessel can be improved.

### Modification of Embodiments

The disclosed embodiments are not limited to the above embodiments, can be embodied in various aspects within a scope not departing from the spirit thereof, and can be modified as follows, for example.

### Modification 1

In the first to seventh embodiments described above, configurations of the lesion retrieval devices 1 and 1A to 1F have been described. However, the configuration of the base wire rod can be modified in various ways. For example, the shape of the transverse cross section of the base wire rod is not limited to a rectangular shape and may be a polygonal shape. The polygonal shape includes a triangular shape, a pentagonal shape, a hexagonal shape, and the like. As described above, the angulated portion of the transverse cross section of the base wire rod contributes to shredding of a tissue fragment since the angulated portion of the transverse cross section of the base wire rod becomes a portion having a convex shape on the surface of the winding wire rod when the base wire rod is twisted, and the angulated portion is sharpened through extension along the twisting direction when the base wire rod is twisted.

For example, the shaft may be a single thread coil in which one wire is wound in a single thread manner. In addition, the shaft may not be a coil and may be a hollow rod-shaped member. Further, the shaft may not be a hollow member, may be a solid member, and may be a metal tube such as a hypotube. Incidentally, when a solid member or a hypotube is used as the shaft, because such a member tends to have high stiffness, a weak portion is preferably provided by, for example, forming a slit using a laser so as to adjust stiffness. In addition, the outer cylindrical tube may be a hollow cylindrical member formed by a single wire dense wound coil or a dense wound coil in which a plurality of wires is spirally wound, or may be a metal tube such as hypotube, for example. In addition, the motor may be omitted, and a handle part capable of manually rotating the shaft may be provided instead of the motor.

For example, the winding pitch of the spirally-arranged protruding portion may not be constant, unlike the illustrated lesion retrieval devices 1 and 1A to 1F, and the winding pitch on the distal end side of the shaft may be different from the winding pitch on the proximal end side of the shaft. As an example, the winding pitch may be designed such that the winding pitch is increased on the distal end side of the shaft to increase efficiency of suctioning a tissue fragment, and the winding pitch is decreased on the proximal end side of the shaft to increase opportunity for contact between a tissue fragment and the convex-shaped portion forming an edge on a surface of the spirally-arranged protruding portion. On the contrary, the winding pitch may be decreased on the distal end side of the shaft, and the winding pitch may be increased on the proximal end side of the shaft.

For example, the spirally-arranged protruding portion may be formed by winding, in the S twist direction around the shaft 10, the winding wire rod that has a configuration in which the base wire rod is twisted in the Z twist direction. Conveyance of a tissue fragment toward the proximal end side can be facilitated also in such a configuration.

For example, the spirally-arranged protruding portion may be formed by winding, in the S twist direction around the shaft 10, the winding wire rod that has a configuration in which the base wire rod is twisted in the S twist direction. Shredding of a tissue fragment can be facilitated also in such a configuration.

For example, the direction relationship between the direction in which the base wire rod is twisted and the winding direction in which the winding wire rod is wound around the shaft in the spirally-arranged protruding portion may not be constant, unlike the illustrated lesion retrieval devices 1 and 1A to 1F, and the direction relationship on the distal end side of the shaft may be different from the direction relationship on the proximal end side of the shaft. The direction relationship herein includes a relationship in which the twisting direction differs from the winding direction or a relationship in which the twisting direction coincides with the winding direction. As an example, the proportion, in the whole length of the spirally-arranged protruding portion, of a portion in which the twisting direction coincides with the winding direction may be increased as the hardness of a lesion tissue to be a target of the lesion retrieval device increases. This is because shredding of a tissue fragment is facilitated in such a portion. In addition, when the lesion retrieval device targets a lesion tissue from which a large amount of tissue fragments are supposed to be dispersed, the proportion, in the whole length of the spirally-arranged protruding portion, of a portion in which the twisting direction differs from the winding direction may be increased. This is because conveying of a tissue fragment to the proximal end side is facilitated in such a portion.

For example, the irregularities on the surface on a transverse cross section of the winding wire rod may not be formed by spirally twisting the base wire rod, but may be formed on the peripheral surface of the wire rod through pressing using a die for molding irregularities, for example.

### Modification 2

The configurations of the lesion retrieval devices 1 and 1A to 1F of the first to seventh embodiments and each of the configurations of the modification 1 described above may be combined, as appropriate. For example, in the lesion retrieval device 1B of the third embodiment, the proximal end side joint part 33d described for the fifth embodiment may be employed and the packing 43 may be omitted, the large diameter portion 24e described for the sixth embodiment may be employed, and the plurality of openings 26f described for the seventh embodiment may be employed. In the lesion retrieval device 1C of the fourth embodiment, the proximal end side joint part 33d described for the fifth embodiment may be employed and the packing 43 may be omitted. In the lesion retrieval device 1D of the fifth embodiment, the large diameter portion 24e described for the sixth embodiment may be employed, and the plurality of openings 26f described for the seventh embodiment may be employed. In the lesion retrieval device 1E of the sixth embodiment, the plurality of openings 26f described for the seventh embodiment may be employed. In the lesion retrieval device 1F of the seventh embodiment, the cutter assembly 35b described for the third embodiment may be employed, and the large diameter portion 24e described for the sixth embodiment may be employed.

Although the present aspects have been described based on the embodiments and the modifications, the embodiments of the above-described aspects are made for facilitating understanding of the present aspects, and do not limit the present aspects. The present aspects can be modified and improved without departing from the spirit thereof and the scope of the claims, and the present aspects include equivalents thereof. Moreover, components may be omitted, as appropriate, unless the technical features thereof are described as essential in the present specification.

What is claimed is:

1. A lesion retrieval device comprising:
a shaft having an elongated outer shape;
a spirally-arranged protruding portion spirally protruding from an outer peripheral surface of the shaft; and
an outer cylindrical tube having a hollow cylindrical shape and accommodating, inside thereof, part of a proximal end side of the shaft provided with the spirally-arranged protruding portion,
wherein:
the spirally-arranged protruding portion comprises a wire rod that is spirally wound around the outer peripheral surface of the shaft, and
a surface of the wire rod in a transverse cross section has irregularities.

2. The lesion retrieval device according to claim 1, wherein:
the wire rod has a structure in which a base wire rod having a polygonal or elliptical transverse cross section is spirally twisted, and
a direction in which the base wire rod is twisted is different from a winding direction in which the wire rod is wound around the shaft.

3. The lesion retrieval device according to claim 1, wherein:
the wire rod has a structure in which a base wire rod having a polygonal or elliptical transverse cross section is spirally twisted, and
a direction in which the base wire rod is twisted is identical to a winding direction in which the wire rod is wound around the shaft.

4. The lesion retrieval device according to claim 1, wherein
the shaft is a single thread coil, or is a multi-thread coil.

5. The lesion retrieval device according to claim 4, wherein
the shaft has, inside thereof, a device lumen into which a medical device is insertable.

6. The lesion retrieval device according to claim 1, further comprising:
a rotation transmission mechanism connected to the shaft,
wherein the rotation transmission mechanism is configured to transmit rotational force to the shaft, thereby rotating the shaft and the spirally-arranged protruding portion around an axis.

7. An atherectomy device comprising:
the lesion retrieval device according to claim 1; and
a cutter provided at a distal end portion of the shaft of the lesion retrieval device, the cutter being configured to excise a body tissue.

8. The lesion retrieval device according to claim 2, wherein
the shaft is a single thread coil, or is a multi-thread coil.

9. The lesion retrieval device according to claim 2, further comprising:
a rotation transmission mechanism connected to the shaft,
wherein the rotation transmission mechanism is configured to transmit rotational force to the shaft, thereby rotating the shaft and the spirally-arranged protruding portion around an axis.

10. An atherectomy device comprising:
the lesion retrieval device according to claim 2; and
a cutter provided at a distal end portion of the shaft of the lesion retrieval device, the cutter being configured to excise a body tissue.

11. The lesion retrieval device according to claim 3, wherein
the shaft is a single thread coil, or is a multi-thread coil.

12. The lesion retrieval device according to claim 3, further comprising:
a rotation transmission mechanism connected to the shaft,
wherein the rotation transmission mechanism is configured to transmit rotational force to the shaft, thereby rotating the shaft and the spirally-arranged protruding portion around an axis.

13. An atherectomy device comprising:
the lesion retrieval device according to claim 3; and
a cutter provided at a distal end portion of the shaft of the lesion retrieval device, the cutter being configured to excise a body tissue.

14. The lesion retrieval device according to claim 4, further comprising:
a rotation transmission mechanism connected to the shaft,
wherein the rotation transmission mechanism is configured to transmit rotational force to the shaft, thereby rotating the shaft and the spirally-arranged protruding portion around an axis.

15. An atherectomy device comprising:
the lesion retrieval device according to claim 4; and
a cutter provided at a distal end portion of the shaft of the lesion retrieval device, the cutter being configured to excise a body tissue.

16. The lesion retrieval device according to claim 5, further comprising:
a rotation transmission mechanism connected to the shaft,
wherein the rotation transmission mechanism is configured to transmit rotational force to the shaft, thereby rotating the shaft and the spirally-arranged protruding portion around an axis.

17. An atherectomy device comprising:
the lesion retrieval device according to claim 5; and
a cutter provided at a distal end portion of the shaft of the lesion retrieval device, the cutter being configured to excise a body tissue.

18. An atherectomy device comprising:
the lesion retrieval device according to claim 6; and
a cutter provided at a distal end portion of the shaft of the lesion retrieval device, the cutter being configured to excise a body tissue.
